SEQUENCE LISTING (6 sequences)

SEQ ID NO: 1 - DNA, 63 bp, Artificial Sequence
  Synthetic CD8 signaling peptide sequence
atgctcaggctgctcttggctctcaacttattcccttcaattcaagtaacaggagggtcttcg

SEQ ID NO: 2 - DNA, 375 bp, Artificial Sequence
  Synthetic EGFR antibody sequence
ggacaggttaaattagaggagtcgggtggtggcagcgtgcagacgggtggtagtctgcgtttgacttgtgctgcctccggccgcacgtcgcgttcttacggtatgggctggttccgccaggctccaggcaaggaacgtgaatttgtatccggcatcagttggcgtggggactcaacaggttacgcagacagcgtcaaaggtcgtttcaccatttcccgcgacaatgcgaagaatacagtcgacttgcagatgaactccctgaaaccagaagatacagctatctactattgtgctgccgcagccggatcagcatggtatggaacattgtacgaatatgactattggggccagggtactcaagtgacggtatctagc

SEQ ID NO: 3 - DNA, 135 bp, Artificial Sequence
  Synthetic CD8 hinge sequence
accacaacgcccgctcctcggccaccgacgccagcgccaactattgcgagtcagcctctcagtctgcgacctgaggcttgtcgaccagcagccggaggcgcagtgcacacgagggggctggacttcgcctgtgat

SEQ ID NO: 4 - DNA, 585 bp, Artificial Sequence
  Synthetic The CD28 transmembrane domain, 4-1BB intracellular domain, and CD3-zeta intracellular domain sequence
agaagacctccttctaagcccttttgggtgctggtggtggttggtggagtcctggcttgctatagcttgctagtaacagtggcctttattattttctgggtgaggaaacggggcagaaagaaactcctgtatatattcaaacaaccatttatgagaccagtacaaactactcaagaggaagatggctgtagctgccgatttccagaagaagaagaaggaggatgtgaactggctagcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgctagc

SEQ ID NO: 5 - DNA, 20 bp, Artificial Sequence
  Synthetic EGFR CAR F
agcgtcaaaggtcgtttcac

SEQ ID NO: 6 - DNA, 20 bp, Artificial Sequence
  Synthetic EGFR CAR R
ggctgactcgcaatagttgg

(19) United States
(12) Patent Application Publication
(10) Pub. No.: US 2024/0000937 A1
(43) Pub. Date: Jan. 4, 2024

(54) METHODS AND COMPOSITIONS OF CAR-EXPRESSING NATURAL KILLER CELLS WITH BISPECIFIC ANTIGEN-BINDING MOLECULES AS CANCER THERAPEUTIC AGENTS

(71) Applicant: World Biotech Regenerative Medical Group Limited, Hong Kong SAR (CN)

(72) Inventors: Wing Yin Cornelia MAN, Hong Kong SAR (CN); Wei MA, Hong Kong SAR (CN); Quangui GAO, Hong Kong SAR (CN)

(21) Appl. No.: 18/027,585

(22) PCT Filed: Sep. 22, 2021
(86) PCT No.: PCT/IB2021/058660

§ 371 Date: Mar. 21, 2023

Related U.S. Application Data
(60) Provisional application No. 63/081,751, filed on Sep. 22, 2020.

(51) Int. Cl. A61K 39/00; A61P 35/00

(52) U.S. Cl. CPC A61K 39/4613; A61P 35/00; A61K 39/4644; A61K 39/4631

(57) ABSTRACT

Provided are a cancer-antigen-specific Natural Killer (NK) cell including a non-viral expression plasmid encoding a chimeric antigen receptor, wherein the chimeric antigen receptor comprises a cancer-antigen-specific single-chain variable fragment (scFv), a hinge region, a transmembrane domain, and intracellular domains; methods of generating said cancer-antigen-specific NK cell; a bispecific antigen-binding molecule comprising a first antigen-binding molecular and a second antigen-binding molecular, wherein the first antigen-binding molecular is an scFv specific to a cancer antigen, and the second antigen-binding molecule is specific to a second cancer antigen and an NK cell receptor, and comprises at least one of an scFv and and an aptamer-based molecule; pharmaceutical compositions comprising at least one of the cancer-antigen-specific NK cell and the bispecific antigen-binding molecule; and methods of treating cancer patients using the pharmaceutical compositions.

### FIELD OF THE DISCLOSURE

The present disclosure relates to cancer-antigen-specific NK cells, bispecific antigen-binding molecules, pharmaceutical combinations of the same, and methods of generating the same and treating cancer patients with same.

### BACKGROUND

Natural killer (NK) cells are a component of an innate immune system and play a major role in the host-rejection of both tumors and virally infected cells. They are an important effector cell type for adoptive cancer immunotherapy. Similar to T cells, NK cells can also be modified to express chimeric antigen receptors (CARs) to enhance anti-tumor activity.

In recent years, research on the CAR has mainly focused on T cells. T cells can be activated and then incubated with vectors encoding the CAR molecules. After incubating for several days, upon cell entry, vectors express the CAR genetic information as RNA which is then reverse transcribed into DNA and permanently integrated into the T cells' genomes. The most commonly reported vector used for CAR T-cell clinical trials are lentivirus vectors.

Although a growing number of CAR-T cell therapies are being developed and tested in clinical trials, their wide clinical application is limited by inherent risks such as graft-versus-host disease (GvHD), tumor lysis syndrome, off-target side effects, and cytokine release syndrome (CRS). To prevent these drawbacks, CAR technology has recently been applied to other immune cells such as natural killer (NK) cells. NK cells can be more easily modified to avoid treatment-related toxicity and immune-mediated adverse effects. CAR-NK cell therapy is generally considered safer than CAR-T cell therapy and is less likely to cause CRS. Haploidentical allogenic NK cell transplantation is reported to almost never induce graft versus host disease (GvHD). In addition, NK cells can be isolated from many sources including peripheral blood and umbilical cord blood. NK cells are also reported to promote dendritic cells to migrate into tumors and enhance anti-PD-1 immunotherapy.

Since rituximab was first approved for the treatment of Non-Hodgkin's lymphoma, antibody-based therapy has been revitalized as a cancer therapeutic. High-specificity, low off-target effects, desirable pharmacokinetics, and high success rates make antibodies amenable for development as drugs. Both pre-clinical studies and early phase clinical trials have demonstrated that bispecific antibody therapy may be an effective treatment for use against solid tumors.

### SUMMARY

The present disclosure relates, in some embodiments, to a cancer-antigen-specific Natural Killer (NK) cell including a non-viral expression plasmid (e.g., pCAR, pT2, pT2B) encoding a chimeric antigen receptor having: a cancer-antigen-specific single-chain variable fragment (scFv); a hinge region; a transmembrane domain; and intracellular domains. In some embodiments, a cancer-antigen-specific NK cell may have a reduced non-specific cytotoxic effect on antigen-negative cells as compared to antigen-expressing cells, an increased specific cytotoxic effect on antigen-expressing tumor cells compared to the cytotoxic effect of an unmodified NK cell, or both.

According to some embodiments, the present disclosure further relates to bispecific antigen-binding molecule including a first antigen-binding molecule having an scFv specific to a first cancer antigen, and a second antigen-binding molecule having at least one of an scFV and an aptamer-based molecule, where the second antigen-binding molecule is specific to a second cancer antigen and an NK cell receptor.

The present disclosure further relates to pharmaceutical compositions including a cancer-antigen-specific Natural Killer (NK) cell and a bispecific antigen-binding molecule.

According to some embodiments, the present disclosure relates to methods for treating a cancer patient including administering to the patient a therapeutically effective amount of at least one of a cancer-antigen specific NK cell, and a bispecific-based antibody, or a pharmaceutical composition including the same. The methods for treating a cancer patient may include cancer patients having at least one of triple negative breast cancer, lung cancer, breast cancer, prostate cancer, glioma, thyroid cancer, colorectal cancer, head and neck cancer, stomach cancer, liver cancer, pancreatic cancer, renal cancer, urothelial cancer, testicular cancer, cervical cancer, endometrial cancer, ovarian cancer, melanoma, esophagogastric cancer.

The present disclosure further relates to methods of producing a cancer-antigen-specific NK cell, the method including: transfecting an NK cell using a non-viral expression plasmid, and inducing an iCaspase-9 gene system. In some embodiments, the non-viral expression plasmid encodes a fusion gene having a cancer-antigen-specific single-chain variable fragment (scFv); a hinge region; a transmembrane domain; and an intracellular domain.

### Definitions

“Antigen” refers to one or more proteins up-regulated in a patient body and they are present in the body fluids of the individual.

“Bispecific antibody” refers to describe a large family of molecules designed to recognize two different epitopes or antigens.

“CD27” is a member of the TNF receptor superfamily. It is currently of interest to immunologists as a co-stimulatory immune checkpoint molecule.

“CD28” (Cluster of Differentiation 28) is one of the proteins expressed on T cells that provide co-stimulation signals required for T cell activation and survival. It is the receptor for CD80 (B7.1) and CD86 (B7.2) proteins. Additionally, it is the only B7 protein receptor constitutively expressed on naive T cells.

“CD40” is a costimulatory protein found on APCs and is required for their activation. The binding of CD154 (CD40L) on T helper (Th) cells to CD40 activates APCs and induces a variety of downstream effects. Deficiency can cause Hyper-IgM syndrome type 3.

“CD133 antigen”, also known as prominin-1, is a glycoprotein that in humans is encoded by the PROM1 gene. It is a member of pentaspan transmembrane glycoproteins, which specifically localize to cellular protrusions.

“Carcinoembryonic antigen” (CEA) describes a set of highly related glycoproteins involved in cell adhesion. CEA is normally produced in gastrointestinal tissue during fetal development, but the production stops before birth.

“Chimeric antigen receptor” refers to the receptor proteins that have been engineered to give NK cells the new ability to target a specific protein. The receptors are chimeric because they combine both antigen-binding and NK-cell activating functions into a single receptor.

“Cytotoxic T-lymphocyte-associated protein 4” (CTLA-4), also known as “Cluster of differentiation 152” (CD152) is a protein receptor that functions as an immune checkpoint. CTLA-4 also downregulates the immune system. It is constitutively expressed in Regulatory T-cells and is upregulated in conventional T-cells after activation, which is a phenomenon notable in cancers.

“Epidermal growth factor receptor” (EGFR) is a transmembrane protein receptor for members of the epidermal growth factor family (EGF family) of extracellular protein ligands.

“Epidermal growth factor receptor variant III” (EGFRvIII) is the most common extracellular domain mutation of EGFR and this mutation leads to a deletion of exons 2-7 of the EGFR gene and renders the mutant receptor incapable of binding any known ligand.

“Epithelial cell adhesion molecule” (EpCAM) is a transmembrane glycoprotein mediating Ca²⁺-independent homotypic cell-cell adhesion in epithelia. EpCAM is also involved in cell signaling, migration, proliferation and differentiation.

“GD2” is a disialoganglioside expressed on tumors of neuroectodermal origin, including human neuroblastoma and melanoma, with highly restricted expression on normal tissues, principally to the cerebellum and peripheral nerves in humans.

“G-protein-coupled receptor (GPCR)-kinase-interacting proteins” (GIT) are expressed by all mammals and birds studied to date and are very highly conserved across mammalian and avian species. Mammals and birds express two GIT proteins: GIT1 and GIT2.

“Glypican-3” (GPC3) is a cell-surface heparin sulfate proteoglycan that binds to the cell surface membrane through a glycosyl-phosphatidylinositol anchor. It expresses predominantly during development, tissue- and stage-specifically in the fetal liver, kidney, vertebrae, and genital system, but not in most of the mature normal organs.

“Herpesvirus entry mediator” (HVEM) (Herpesvirus entry mediator) also known as TNF receptor superfamily member 14 (TNFRSF14), is a human cell surface receptor of the TNF-receptor superfamily.

“Human epidermal growth factor receptor 2” (HER2) is a membrane tyrosine kinase and oncogene that is overexpressed and gene amplified in about 20% of breast cancers.

“Inducible T Cell Costimulator” (ICOS) is a protein coding gene. Diseases associated with ICOS include Immunodeficiency common variable 1 and Common Variable Immunodeficiency. Among its related pathways are downstream signaling events of B cell receptor and GAB1 signalosome.

“Interleukin 12 receptor” (IL-12 receptor) is a Type 1 cytokine receptor binding IL-12. It consists of an IL-12 receptor beta-1 subunit and an IL-12 receptor beta-2 subunit.

“Interleukin-18 receptor” (IL-18) is an interleukin-receptor of the immunoglobulin superfamily..

“Lymphocyte-activation gene 3” (LAG-3) (Lymphocyte-activation gene 3) is a protein which in humans is encoded by the LAG3 gene. LAG-3 is a cell surface molecule having diverse biologic effects on T-cell function. LAG-3 is an immune checkpoint receptor that is the target of various drug development programs seeking to develop new treatments for cancer and autoimmune disorders.

“Mesothelin” is a tumor differentiation antigen that is normally present on the mesothelial cells lining the pleura, peritoneum, and pericardium. It is highly expressed in several human cancers including malignant mesothelioma, pancreatic cancer, ovarian cancer, and lung adenocarcinoma.

“Mucin 1” (MUC1) is a glycoprotein with extensive O-linked glycosylation of its extracellular domain. Mucins line the apical surface of epithelial cells in the lungs, stomach, intestine, eyes, and several other organs.

“Mucin 16” (MUC16), previously known as CA125, is a protein that in humans is encoded by the MUC16 gene. It is a member of the Mucin family of glycoproteins.

“Natural killer group 2D” (NKG2D) is a C-type, lectin-like, type II transmembrane glycoprotein whose transcript was initially discovered in human NK and T cells.

“0X40” is a secondary co-stimulatory immune checkpoint molecule, expressed after 24 to 72 hours following activation; its ligand, OX40L, is also not expressed on resting antigen presenting cells, but is following their activation.

“Programmed cell death-1” (PD-1) is a member of the CD28 superfamily that delivers negative signals upon interaction with its two ligands, PD-L1 or PD-L2.

“Programmed death-ligand 1” (PD-L1), also known as cluster of differentiation 274 (CD274) or B7 homolog 1 (B7-H1), is a human protein that is encoded by the CD274 gene. PD-L1 is overexpressed on tumor cells and on non-transformed cells in the tumor microenvironment. In addition, PD-L1 that is expressed on the tumor cells will bind to PD-1 receptors that are located on activated T cells. This binding event leads to the inhibition of the cytotoxic T cells.

“Prostate stem cell antigen” (PSCA) is a protein that in humans is encoded by the PSCA gene. This gene encodes a glycosylphosphatidylinositol-anchored cell membrane glycoprotein. In addition to being highly expressed in the prostate it is also expressed in the bladder, placenta, colon, kidney, and stomach.

“Prostate-specific membrane antigen” (PSMA) is considered as one of the most successful targets for imaging and therapy in nuclear medicine. It is a glycoprotein, a membrane-bound metallo-peptidase, encoded by FOLH1 gene on chromosome 11.

“T cell immunoreceptor with Ig and MM domains” (TIGIT) is an immune receptor present on some T cells and Natural Killer cells (NK). It is also identified as WUCAM and Vstm3. TIGIT may bind to CD155 (PVR) on dendritic cells (DCs) and macrophages with high affinity. Additionally, TIGIT may also bind to CD112 (PVRL2), but with lower affinity than it binds to PVR and DCs.

“T-cell immunoglobulin and mucin domain 1” (TIM-1), also known as Kim-1 or HAVcr1, a receptor for Zaire Ebolavirus and Lake Victoria Marburgvirus and a type 1 transmembrane protein composed of an extracellular Ig variable (IgV) domain, a mucin-like domain, a transmembrane domain, and a cytoplasmic tail. The protein is expressed on dividing cells of the kidney epithelium and on activated type 2 helper T cells (Th2 cells), and binds to phosphatidylserine (PS) as well as family member TIM-4, resulting in the clearance of apoptotic cells and activation of T-cell proliferation, respectively.

“T-cell immunoglobulin and mucin-domain containing-3” (TIM-3), also known as Hepatitis A virus cellular receptor 2 (HAVCR2), is a protein that in humans is encoded by the HAVCR2 gene.

“4-1BB” (also known as CD137) is a type 2 transmembrane glycoprotein receptor belonging to the tumor necrosis factor superfamily, expressed on activated T-lymphocytes. 4-1BBL (4-1BB ligand, CD137L) is found on antigen presenting cells (APCs) and binds to CD137.

### DETAILED DESCRIPTION

The present disclosure encompasses and is drawn to cancer-antigen-specific NK cells, particularly those that have a reduced non-specific cytotoxic effect on antigen-negative cells and/or increased specific cytotoxic effect on antigen-expressing tumor cells, and the generation thereof. The present disclosure further encompasses bispecific antigen-binding molecules. The disclosed cancer-antigen-specific NK cell may be combined with the bispecific antigen-binding molecules to for a pharmaceutical composition that can be used to treat cancer.

### A. Cancer-Antigen-Specific Natural Killer (NK) Cells

The present disclosure relates, in some embodiments to a cancer-antigen-specific NK cell. A cancer-antigen-specific NK cell of the present disclosure may include a non-viral expression plasmid encoding a chimeric antigen receptor. The use of a non-viral CAR construct for the CAR-NK may increase safety and avoid cellular cytotoxicity and genotoxicity which may be caused by conventional lentiviral or retroviral integrants. In some embodiments, a CAR construct may be inserted into a modified non-viral expression sleeping beauty (SB) transposon plasmid backbone. Examples of SB transposon vector plasmids include, but are not limited to, pT2, pT2B, and pT3. In some embodiments, the SB transposon platform may be modified in a way so the transfection efficiency into the NK cells will be increased (e.g. vectorizing the SB components into minicircle DNA).

The non-viral SB expression plasmid may be transfected into NK cells. In some embodiments, transfection may be performed using at least one of electroporation, nanoparticles, calcium phosphate, and lipofection. Upon entry into the NK cell, the CAR fusion gene may integrate into the NK cell genome.

According to some embodiments, a chimeric antigen receptor may have: (a) a cancer-antigen-specific single-chain variable fragment (scFv); (b) a hinge region; (c) one or more transmembrane domains; and (d) one or more intracellular domains.

A target antigen-specific scFv antibody fragment may include a fusion protein of the variable regions of the heavy (VH) and light chains (VL) of antibodies. This fragment is responsible for specific binding of the CAR to the target antigen on a tumor cell. For example, a scFv fragment developed from a GPC3 antibody may guide the CAR to bind specifically to the GPC3-expressing cancer cells.

A hinge region may be an extracellular structural region of the CAR that separates the scFv from the transmembrane domain. The hinge region may have responsible for supplying stability for efficient CAR expression and activity.

A transmembrane domain may be a hydrophobic a (alpha) helix domain which anchors the CAR in the cell membrane. A transmembrane domain (TMD) may be a conserved motif that shares homology with other intracellular domains such as CD3ζ (zeta). In one embodiment, the transmembrane domain may be a CD28-encoded transmembrane region. Intracellular domains provide co-stimulatory signals to the NK cells when the CAR receptor binds to its target molecule. The stimulatory signal may promote NK cell activation, proliferation and survival.

In some embodiments, an intracellular domains may include OX40, 4-1BB, CD3ζ (zeta), or a combination thereof. According to some embodiments, the TMD may be CD28, the intracellular signaling domain may be CD3ζ (zeta) and the vector is integrated.

In some embodiments, the NK cells of the present disclosure may be characterized by PCR analysis of their genomic DNA. Vector integrations in a NK cell clone may be mapped by linear amplification-mediated PCR and DNA sequencing. To amplify products of defined length and sequence, PCR analysis of the genomic DNA may be performed with oligonucleotide primers hybridizing to genomic and vector DNA sequences adjacent to 5′ and 3′ junction sites of chromosomal and integrated vector sequences. Additionally, vector integrations may be confirmed to molecularly identify the cell clone NK of the present disclosure.

According to some embodiments, a cancer-antigen-specific NK cell may have a reduced non-specific cytotoxic effect on antigen-negative cells as compared to antigen-expressing cells, an increased specific cytotoxic effect on antigen-expressing tumor cells compared to the cytotoxic effect of an unmodified NK cell, or both. A reduction in non-specific cytotoxicity may constitute an important safety feature of the NK cells from a clinical perspective. The NK cells of the present disclosure may exert high efficiency on target antigen (e.g., EGFR) expressing tumor cells in contrast to unmodified NK cells. However, in comparison to unmodified NK cells, the disclosed NK cells may be less efficient in attacking target antigen-negative, non-target cells.

To further increase the safety of the therapeutics when encountering adverse effects in cancer patients, an inducible suicide gene Caspase9 (iCasp9) may be incorporated into the vector. The incorporation represents a safety induction switch that allows removal of excessively activated CAR-NK cells. Induction of iCasp9 may involve the administration of the small molecule drug AP1903, which can induce dimerization and subsequent apoptosis of the transduced cells.

### B. Bi-Specific Aptamer Based Antibodies

The present disclosure further relates to bispecific antigen-binding molecules specific for cancer-antigens and NK cell receptors. In some embodiments, bispecific antigen-binding molecules may contain a first antigen-binding molecule including a first single-chain variable fragment (scFv) having specificity for one cancer specific antigen and a second antigen-binding molecule that may be a second scFv specific for an NK cell receptor. In another embodiment, one or both of the antigen-binding molecules of the bispecific antigen-binding molecule may be an aptamer-based molecule that may function as an antibody against a second tumor antigen and has specificity for one or more receptors on NK cells. In some embodiments, a first scFv may include a first variable region of heavy chain (VH) and a first variable region of light chain (VL). A second scFv may include a second VH and a second VL. In some embodiments, a second scFv may be replaced by an aptamer-based tertiary structure mimicking the function of the bispecific antigen-binding molecule against the second tumor antigen. The bispecific antigen-binding molecules of the present disclosure may further enhance the activation signal for the NK cells. The generated CAR-NK bispecific therapeutics may only attack target antigen-positive cancer cells. A cancer specific antigen may include any one or more of CD133, GD2, MUC1, EpCAM, PSCA, HER2, and MUC16, in some embodiments. According to some embodiments, the NK cell receptor may be selected from: NKG2D, 4-1BB, OX40, CD27, CD40, TIM-1, CD28, HVEM, GIT, ICOS, IL12 receptor, IL18 receptor, PD-1, TIM-3, LAG-3, TIGIT, CTLA-4, and PD-L1.

### C. Pharmaceutical Compositions

The present disclosure further relates to a pharmaceutical composition having at least one of cancer-antigen-specific NK cells and bispecific antigen-binding molecules. The cancer-antigen-specific NK cell may contain a non-viral expression plasmid encoding a chimeric antigen receptor. The chimeric antigen receptor may have: (a) a cancer-antigen-specific single-chain variable fragment (scFv); (b) a hinge region; (c) a transmembrane domain; and (d) one or more intracellular domains, as described in the present application. The cancer-antigen-specific NK cell may also have a reduced non-specific cytotoxic effect on antigen-negative cells as compared to antigen-expressing cells, an increased specific cytotoxic effect on antigen-expressing tumor cells compared to the cytotoxic effect of an unmodified NK cell, or both.

The bispecific antigen-binding molecule may have a first antigen-binding molecule specific for a first cancer antigen and a second antigen-binding molecule specific to a second cancer antigen, an NK cell receptor, or both.

A disclosed pharmaceutical composition may include a combination of CAR-expressing NK cells and bispecific antigen-binding molecules for targeting antigen-expressing solid tumors. In some embodiments, a bispecific antigen-binding molecule may have specificity for both a cancer-specific antigen and an NK cell receptor. An NK cell receptor may include NKG2D, 4-IBB, OX40, CD27, CD40, TIM-1, CD28, HVEM, GIT, ICOS, IL12 receptor, IL18 receptor, PD-1, TIM-3, LAG-3, TIGIT, CTLA-4, and PD-L1. A cancer-specific antigen may include CD133, GD2, MUC1, EpCAM, PSCA, HER2, and MUC16.

### D. Method of Treatment and Indications

As described above, the present disclosure relates to pharmaceutical compositions containing cancer-antigen-specific NK cells and bispecific antigen-binding molecules. The disclosed pharmaceutical compositions may be used in the prevention and/or treatment of cancer, including target antigen (e.g., EGFR, EGFRvIII) expressing cancers, by administering to the patient a therapeutically effective amount of cancer-antigen-specific NK cells and bispecific antigen-binding molecules.

Preferably said treatment method comprises administering to a subject a therapeutically effective amount of: (1) NK cells according to the present invention or NK cells obtained by the method according to the present invention; (2) bispecific antigen-binding molecules according to the present invention; (3) both the NK cells of (1) and the bispecific antigen-binding molecules of (2); and (4) optionally, respective excipients. A “therapeutically effective amount” refers to the amount that is sufficient to treat the respective disease (cancer) or achieve the respective outcome of the adoptive, target-cell specific immunotherapy.

Patients with advanced, refractory, metastasized cancers, or patients treated with chemotherapies may experience NK cell exhaustion. For this reason, only if the cancer patient fulfills the criteria of having complete blood count (CBC)>4×10⁹/L, Neutrophils/Lymphocytes<5, and Lymphocytes>20%, and tests negative for a panel of four infectious diseases (HIV, HBV, HCV, Syphilis) may NK cells be isolated from the peripheral blood of that cancer patient. If the patient does not meet the criteria and/or tests positive for any of the screened infectious diseases, the NK cells from umbilical cord blood or NK-92 cell line may be used. In some embodiments, haploidentical NK cells can be used after HLA-haplotyping with potential donors such as siblings or descendants. To ensure>99% purity of the haploidentical NK cells, T-cell depletion will be performed on day 1 and day 14 of the NK cell culture process.

Cancers that may be treated by the disclosed cancer-antigen-specific NK cells and bispecific antigen-binding molecules may include, but are not limited to, cancers that express EGFR, EGFRvIII, mesothelin, NKG2D, CEA, PSMA, and GPC3. An EGFR-expressing cancer may include triple negative breast cancer, lung cancer, breast cancer, prostate cancer, glioma, thyroid cancer, colorectal cancer, head and neck cancer, stomach cancer, liver cancer, pancreatic cancer, renal cancer, urothelial cancer, testis cancer, cervical cancer, endometrial cancer, ovarian cancer, melanoma, and esophagogastric cancer. An EGFRvIII-expressing cancer may include glioblastoma and brainstem glioma. A mesothelin-expressing cancer may include mesothelioma, lung cancer, pancreatic cancer, and ovarian cancer. A NKG2D ligand-expressing cancer may include Ewing's sarcoma and ovarian cancer. A CEA-expressing cancer may include pancreatic cancer, colorectal cancer, lung cancer, breast cancer, and gastrointestinal cancer. A PSMA-expressing cancer may include prostate cancer. A GPC3-expressing cancer may include embryonal tumors (e.g., Wilms tumor, hepatoblastoma, and neuroblastoma), germ cell tumors (e.g., yolk sac tumor, immature teratoma, and embryonal carcinoma), carcinomas (e.g., hepatocellular carcinoma and pulmonary squamous cell carcinoma), sarcomas (e.g., malignant rhabdoid tumor and rhabdomyosarcomas), and malignant melanoma.

### E. Method of Producing Cancer-Antigen-Specific NK Cells

A disclosed method may include the following steps:

1) Inserting a codon-optimized fusion gene comprising a signaling peptide, a target-specific binding molecule, a hinge region, a transmembrane domain, and intracellular domains into a vector (e.g., SB transposon vector plasmid pT2B) for transfecting NK cells.

2) Transfecting NK cells with the vector. Transfection may be performed using at least one of electroporation, nanoparticles, calcium phosphate, and lipofection. According to some embodiments, transfection may be performed via an optimized electroporation method. The optimized electroporation method may include using electroporation buffer with an osmolality between 0.32-0.45 Osmol/kg, with a pulse voltage of 220-250V, depending on the selected type of NK cells being transfected. In some embodiments, transfecting NK cells may result in a cell viability of 75%-85% (e.g., primary NK cells, cord-blood derived NK cells or NK-92 cells).

3) Generating single cell clones, for example by limiting dilution or flow cytometric single-cell sorting.

4) Identifying CAR-expressing NK cells, for example by flow cytometric analysis or ELISA assay with a target antigen (e.g., EGFR-Fc fusion protein). CAR-expressing single clones may be characterized according to the molecular structure, or the stability, or cytokine production, and the ability to induce cytotoxic effect towards specific antigen-expressing cancer cells.

5) Further selecting a cell clone that displays both high and stable CAR-expression during continuous culture. For example, a desirable clone may display at least a 6-fold shift in Mean Fluorescent Intensity (MFI) in flow cytometric analysis with the shift intensity being largely maintained for an extended time period or across multiple passages (e.g., at least 14-18 days, or across 3-6 passages).

6) Evaluating the cytotoxicity of the retargeted cells against target antigen (e.g., EGFR) expressing cells. Cytotoxicity may be evaluated by any cell viability assay including, but not limited to, FACS-based assays, trypan blue assays, crystal violet assays, and enzyme leakage assays. In some embodiments, the FACS-based cell viability assay may be a double-staining viability assay used to exclude spontaneously lysed target cells in the absence of effector cells.

7) Evaluating cytotoxic activity of the retargeted cells against target antigen (e.g., EGFR) negative cells. Cytotoxicity may be evaluated by any cell viability assay including, but not limited to, FACS-based assays, trypan blue assays, crystal violet assays, and enzyme leakage assays. In some embodiments, the FACS-based cell viability assay may be a double-staining viability assay used to exclude spontaneously lysed cells in the absence of effector cells.

8) Selecting a cell clone that displays at least one of: a high cytotoxicity against target antigen (e.g., EGFR) expressing cells and a low or no cytotoxicity against target antigen-negative cells. In some embodiments, cytotoxicity may be largely detected in target antigen-positive primary NK cells or NK-92 cells with overexpressed target antigens. According to some embodiments specific cytotoxic activities may be at least 6.2 to 12.8 fold higher in target antigens positive cancer cells.

9) Determining the number and position of vector integration using techniques such as linear amplification-mediated PCR and DNA sequencing. In some embodiments, integration sites may be further confirmed by PCR analysis of genomic DNA of cells from different passages during continuous culture. A cell clone exhibiting vector integration in an intergenic region may then be selected.

### EXAMPLES

### Example 1. NK Cell Isolation and Purification from the Peripheral Blood of Cancer Patient

For autologous NK purification and culture, at least 100 mL peripheral blood was drawn from a cancer patient. The peripheral blood drawn from the cancer patient was diluted with PBS at the ratio of 1:1. After then, 15 mL Ficoll®-Paque Premium (GE Healthcare) was used to isolate the peripheral blood mononuclear cells (PBMCs) from each 30 mL diluted blood in SepMate™-50 tube (STEMCELL Technologies, Vancouver, Canada). The PBMCs layer was carefully collected and ACK lysing buffer (ThermoFisher, Waltham, MA, USA) was added to remove the remaining red blood cells. NK cells were isolated and purified from PBMCs with a biotin-antibody cocktail (BioLegend, San Diego, CA) and streptavidin nanobeads (BioLegend, San Diego, CA). Flow cytometry was used to check the NK purity with cell surface marker CD3-and CD56+.

### Example 2. Generation of EGFR-Specific NK Cell

An EGFR-target CAR receptor was designed with an immunoglobulin heavy chain signal peptide (SEQ ID NO. 1), EGFR-specific scFv antibody fragment (SEQ ID NO. 2), a CD8 hinge region (SEQ ID NO. 3), and a hybrid sequence containing a CD28 transmembrane domain, a 4-1BB intracellular domain, and a CD3ζ (zeta) intracellular domain (SEQ ID NO. 4). A codon-optimized fusion gene was synthesized and inserted into the SB transposon pT2B plasmid. Transfection was performed using an optimized electroporation method having an electroporation buffer with an osmolality between Osmol/kg, and a pulse voltage of 220-250V.

### Example 3. Molecular Characterization of EGFR-Specific NK Cell

Linear amplification-mediated PCR and DNA sequencing revealed one vector integration in each integration region of the clonal EGFR-specific NK cells. The integration sites were further confirmed by PCR analysis of the genomic DNA of the EGFR-specific NK cells from three different passages during continuous culture. This amplified specific DNA sequences that encompass the junction between the EGFR gene and the 5′ end of the integrated CAR vector, and between the 3′ end of the integrated CAR vector and EGFR gene.

Additionally, genomic DNA of the different passages of the EGFR-specific NK cells yielded the same characteristic amplification products of defined length and sequence. This demonstrates the long-term stability of the vector integrations. No amplification products were obtained with the same oligonucleotide primers (SEQ ID NO. 5 and 6) upon PCR analysis of genomic DNA from unmodified NK cells. This is indicative of a specific and powerful diagnostic tool that can be used to molecularly identify the EGFR-specific NK cell clone.

### Example 4. Cytotoxicity Assays

Cytotoxicity of NK cells towards target cells was analyzed in FACS-based assays. In brief, the target cells were labeled with calcein violet AM and then co-cultured with effector cells at a variety of effector to target (E/T) ratio for 2 h in a 37° C. incubator. After co-culturing, 250 μL of propidium iodide (PI) solution (1 μg/mL) was added to each sample and incubated for 5 min. followed by performing a flow cytometric analysis in a flow cytometer (BD Bioscience, Heidelberg, Germany). The results were analyzed using FACSDiva software from BD Biosciences. In addition, the 5 number of spontaneously lysed target cells in the absence of effector cells was subtracted from the number of death target cells determined as calcein violet AM and PI double positive in the measured sample to calculate the specific cytotoxicity.

What is claimed is:

1. A cancer-antigen-specific Natural Killer (NK) cell comprising:
a non-viral expression plasmid encoding a chimeric antigen receptor, the chimeric antigen receptor comprising:
a cancer-antigen-specific single-chain variable fragment (scFv);
a hinge region;
a transmembrane domain; and
intracellular domains.

2. The cancer-antigen-specific NK cell of claim 1, wherein at least one of:
the cancer-antigen-specific NK cell has a reduced non-specific cytotoxic effect on antigen-negative cells as compared to antigen-expressing cells, and
the cancer-antigen-specific NK cell has increased specific cytotoxic effect on antigen-expressing tumor cells compared to the cytotoxic effect of an unmodified NK cell.

3. A bispecific antigen-binding molecule comprising:
a first antigen-binding molecule comprising an scFv specific to a first cancer antigen; and
a second antigen-binding molecule comprising at least one of an scFV and an aptamer-based molecule,
wherein the second antigen-binding molecule is specific to a second cancer antigen and an NK cell receptor.

4. A pharmaceutical composition, the pharmaceutical composition comprising:
(a) at least one of:
(i) a first cancer-antigen specific NK cell comprising:
a non-viral expression plasmid encoding a chimeric antigen receptor, the chimeric antigen receptor comprising:
a cancer-antigen-specific single-chain variable fragment (scFv);
a hinge region;
a transmembrane domain; and
intracellular domains; and
(ii) a second cancer-antigen specific NK cell comprising
a non-viral expression plasmid encoding a chimeric antigen receptor, the chimeric antigen receptor comprising:
a cancer-antigen-specific single-chain variable fragment (scFv);
a hinge region;
a transmembrane domain; and
intracellular domains,
wherein at least one of:
the cancer-antigen-specific NK cell has a reduced non-specific cytotoxic effect on antigen-negative cells as compared to antigen-expressing cells, and
the cancer-antigen-specific NK cell has increased specific cytotoxic effect on antigen-expressing tumor cells compared to the cytotoxic effect of an unmodified NK cell; and
(b) a bispecific antigen-binding molecule comprising:
(i) a first antigen-binding molecule comprising an scFv specific to a first cancer antigen; and
(ii) a second antigen-binding molecule comprising at least one of an scFV and an aptamer-based molecule,
wherein the second antigen-binding molecule is specific to a second cancer antigen and an NK cell receptor.

5. A method for treating a patient with cancer, the method comprising administering to the patient a therapeutically effective amount of at least one of:
(a) at least one of:
(i) a first cancer-antigen specific NK cell comprising:
a non-viral expression plasmid encoding a chimeric antigen receptor, the chimeric antigen receptor comprising:
a cancer-antigen-specific single-chain variable fragment (scFv);
a hinge region;
a transmembrane domain; and
intracellular domains; and
(ii) a second cancer-antigen specific NK cell comprising
a non-viral expression plasmid encoding a chimeric antigen receptor, the chimeric antigen receptor comprising:
a cancer-antigen-specific single-chain variable fragment (scFv);
a hinge region;
a transmembrane domain; and
intracellular domains,
wherein at least one of:
the cancer-antigen-specific NK cell has a reduced non-specific cytotoxic effect on antigen-negative cells as compared to antigen-expressing cells, and
the cancer-antigen-specific NK cell has increased specific cytotoxic effect on antigen-expressing tumor cells compared to the cytotoxic effect of an unmodified NK cell; and
(b) a bispecific antigen-binding molecule comprising:
(i) a first antigen-binding molecule comprising an scFv specific to a first cancer antigen; and
(ii) a second antigen-binding molecule comprising at least one of an scFV and an aptamer-based molecule,
wherein the second antigen-binding molecule is specific to a second cancer antigen and an NK cell receptor.

6. The method of claim 5, wherein the cancer is at least one of triple negative breast cancer, lung cancer, breast cancer, prostate cancer, glioma, thyroid cancer, colorectal cancer, head and neck cancer, stomach cancer, liver cancer, pancreatic cancer, renal cancer, urothelial cancer, testicular cancer, cervical cancer, endometrial cancer, ovarian cancer, melanoma, and esophagogastric cancer.

7. A method of producing a cancer-antigen-specific NK cell, the method comprising:
transfecting an NK cell using a non-viral expression plasmid; and
inducing an iCaspase-9 gene system.

8. The method of claim 7 wherein the non-viral expression plasmid encodes a fusion gene comprising:
a cancer-antigen-specific single-chain variable fragment (scFv);
a hinge region;
a transmembrane domain; and
an intracellular domain.
